(19) United States
(12) Patent Application Publication
(10) Pub. No.: US 2017/0157317 A1
(43) Pub. Date: Jun. 8, 2017

(54) Treatment Method to Cure Diseases Within a Patient by Administering Intravenous Oxygen Mixtures

(71) Applicant: Priya Visweswaran Balakrishnan, Houston, TX (US)

(72) Inventor: Priya Visweswaran Balakrishnan, Houston, TX (US)

(21) Appl. No.: 15/373,453

(22) Filed: Dec. 8, 2016

Related U.S. Application Data
(60) Provisional application No. 62/264,522, filed on Dec. 8, 2015.

(51) Int. Cl. A61M 5/14; A61M 39/28; A61M 5/31; A61M 5/168; A61M 5/158; A61B 17/132

(52) U.S. Cl. CPC A61M 5/1407; A61M 5/158; A61B 17/132; A61M 5/31; A61M 5/16831; A61M 39/28; A61M 2005/006

(57) ABSTRACT

Disclosed is a treatment method to cure diseases within a patient by administering intravenous oxygen-ozone mixture.

The current application claims a priority to the U.S. Provisional Patent application Ser. No. 62/264,522 filed on Dec. 8, 2015.

### FIELD OF THE INVENTION

The present invention generally relates to a method of using an ozone-oxygen mixture to treat various diseases. More specifically, the present invention intravenously administers the ozone-oxygen mixture to a patient through the use of a syringe and an infusion device.

### BACKGROUND OF THE INVENTION

The present invention concerns a method of administration of oxygen-ozone mixture to cure disease. Usages of the method disclosed in the present invention include but are not limited to treating conditions such as Chronic Kidney Disease, End Stage Renal Disease, Diabetes, Heart Disease (Coronary Artery Disease, Congestive Heart Failure), Neurological diseases such as neuropathy and strokes and degenerative diseases, including dementia, Parkinsons's disease, ALS and MS, Hypertension, High Cholesterol, COPD, Atrial Fibrillation, Osteoporosis, Osteoarthritis, Asthma, Depression, Hepatitides, and Lyme Disease, genital herpes and HIV. It is also conceivable that the present invention may be used to treat every infectious disease including Ebola, inflammatory and autoimmune diseases, chronic fatigue, and like diseases. The present invention may be used in cancer with variable results.

### BRIEF DESCRIPTION OF THE DRAWINGS

FIG. 1 is a flowchart for the method used to cannulate the veins.

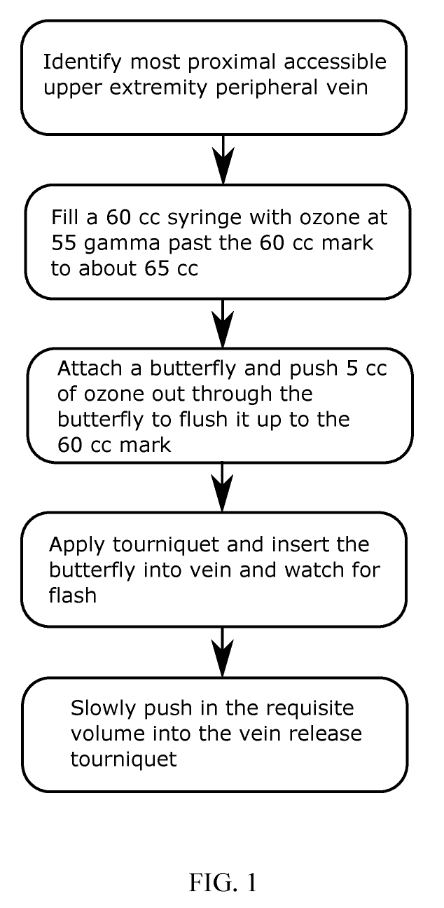

FIG. 2 is a flowchart for the methods comprising a treatment session.

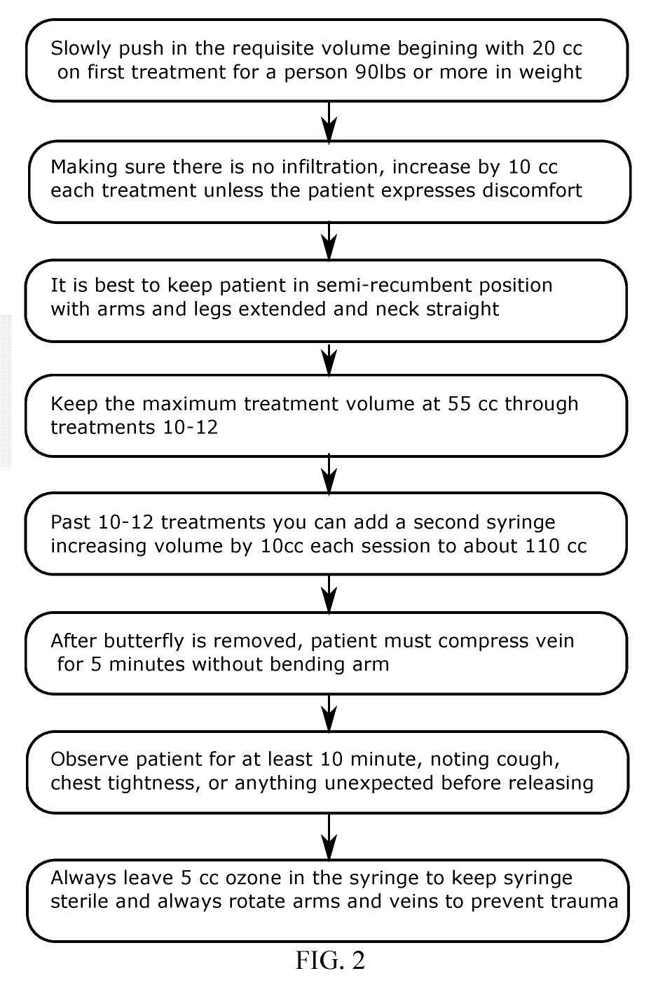

### DETAILED DESCRIPTION OF THE INVENTION

All illustrations of the drawings are for the purpose of describing selected versions of the present invention and are not intended to limit the scope of the present invention.

The present invention is a system and a method to cure diseases with a patient by administering intravenous oxygen-ozone mixture. A proprietary machine has been designed to create this gaseous oxygen-ozone mixture with precision concentration controls and sterility. This gaseous oxygen-ozone mixture is then injected into the patient increasing doses starting with 10 to 20 milliliters depending upon the patient's weight and the patient's habitus. Common contraindications to the usage of this therapeutic mixture include recent (less than 3 months) acute myocardial infarction, pregnancy, and bleeding diathesis and Glucose-6 Phosphate Dehydrogenase enzyme absolute or acquired deficiency (gleaned by eliciting a history of sulfa drug allergy, or a blood test for the enzyme level if the patient has never had a sulfa drug). In addition, an administrator is generally provided with a syringe, an infusion device, and a tourniquet (Step A) in order to complete the overall process of the present invention. In the preferred embodiment, the syringe is configured to retain a volume of 60 mL, and the infusion device is a 27-gauge winged infusion set that is known in the relevant arts as a butterfly. The administrator should also be a licensed medical practitioner.

The overall process of the present invention begins by allowing the administrator to identify an accessible peripheral vein on an upper extremity of the patient (Step B). The upper extremity is preferably the arm or the elbow of patient. The administrator then prepares a volumetric dosage of oxygen-ozone mixture with the syringe (Step C), which is used to retain and intravenously inject the volumetric dosage of oxygen-ozone mixture into the patent. In the preferred embodiment, the a 60-mL syringe is filled with ozone at 55 gamma (55 mcg/mL) past the 60-mL mark to about the 65-mL mark. Also in the preferred embodiment, the oxygen-ozone mixture is approximately composed of 96% oxygen and 4% ozone. The overall process continues as the administrator applies the tourniquet to a cannulation area on the upper extremity and subsequently inserts the infusion device into the accessible peripheral vein (Step D). The tourniquet is applied to the cannulation area in order to improve blood circulation through the veins of the patient, which in turn allows for easier execution of Step E. After the administrator witnesses a flash of blood within the cannulation area, the administrator releases the tourniquet from the cannulation area. This allows the overall process to conclude by transferring the volumetric dosage of oxygen-ozone mixture from the syringe through the infusion device, and into the accessible peripheral vein at a specified infusion rate (Step E). The volumetric dosage of oxygen-ozone mixture can then be slowly infused into the accessible peripheral vein. The preferred infusion rate is approximately 1 mL every 5 to 15 seconds, and the preferred duration for Step E should last one to two minutes. Yet, the proper infusion rate is proportionally dependent on the diametrical size of the accessible peripheral vein. Thus, the administrator can adjust the specified infusion rate to be proportionately slower if the accessible peripheral vein has a smaller diameter.

The present invention also allows the overall process to be repeated between the administrator and the patient as a plurality of treatment sessions. Thus, Step B through Step E is repeated during each treatment sessions. In order for the patient to garner the most health benefits from receiving multiple treatment sessions, the administrator incrementally increases the volumetric dosage of oxygen-ozone mixture by a specified volume during each treatment session. The specified volume is a dosage escalation that is proportionately depended upon a weight, a habitus, and an underlying health condition of the patient. In the preferred embodiment, the volumetric dosage of oxygen-ozone mixture is increased by approximately 10 mL in each successive treatment session. However, the dosage escalation can be reduced or completely removed if the patient is experiencing procedural complications such as Herxheimer reaction, expresses discomfort, cough, chest tightness, facial flush, or vein irritation. Vein discomfort may be mitigated by slowing down the infusion rate. Thus, the specified volume for the dosage escalation should typically range between 0 mL to 10 mL, but no more. In addition, the administrator must also make sure to prevent any infiltration.

During an initial session for the plurality of treatment sessions, the volumetric dosage of oxygen-ozone mixture is within a preferred range of 10 to 20 mL for a 90-or-more pound (lb.) person. The exact volumetric dosage of oxygen-ozone mixture for the initial session is also proportionately depended upon a weight, a habitus, and an underlying health condition of the patient and is preferably determined by the administrator. Consequently, a lighter patient would require a dosage closer to 10 mL, and a heavier patient would require a dosage closer to 20 mL. However, the present invention allows for an even-lower volumetric dosage of oxygen-ozone mixture of 2.5 mL if the patient's weight is proportionately low as well.

From a tenth session to a twelfth session for the plurality of treatment sessions, the volumetric dosage of oxygen-ozone mixture is about 55 mL for an approximately 90-or-more lb. person Likewise, the exact volumetric dosage of oxygen-ozone mixture for these later sessions is proportionately depended upon a weight, a habitus, and an underlying health condition of the patient and is preferably determined by the administrator.

The present invention also allows for different actions to improve the efficiency of Step E. One such action is to have the patient straighten their appendages during Step E, which prevents an obstruction from occurring as the volumetric dosage of oxygen-ozone mixture transfers into the accessible peripheral vein. This means that the patient's appendages should be outstretched and not bent. Another such action is to have the patient resting in a semi-recumbent position in order to improve the efficiency of Step E. In addition, if an obstruction for transferring the volumetric dosage of oxygen-ozone composition is detected during Step E, then another such action that could be taken by the administrator is to stroke the proximal area on the upper extremity towards the heart of the patient. This stroking or “milking” movement by the administrator should relieve the obstruction felt by the patient. Another such action is to have the patient do a weight-lifting exercise to improve circulation around the cannulation area. Another such action is to have the patient drink plenty of liquids and be hydrated before a treatment session.

The present invention also allows the overall process to be simultaneously repeated between the patient and the administrator. Thus, the administrator is able to simultaneously execute a first iteration of Steps A through E with a first syringe and a second iteration of Steps A through E with a second syringe. This allows the administrator to double the volumetric dosage of oxygen-ozone mixture during a single treatment session, while first clamping (with taped jaws) the tubing at the distal end of the intravenous tubing, to prevent air entry, while switching out syringes. Special safety precautions need to be taken by the administrator for the patient while using two syringes. For the plurality of treatment sessions, the simultaneous execution of the first iteration and the second iteration is done during the later treatment sessions. In the preferred embodiment, the simultaneous execution of the first iteration and the second iteration allows the volumetric dosage of oxygen-ozone mixture to range between 110 mL to 120 mL.

Moreover, the infusion device is removed after the complete infusion of the desired dosage, whereupon it is desired that the patient compresses the cannulated vein for at least 5 minutes without bending the body part containing the vein. After the treatment, the administrator observes the patient for at least 10 minutes before allowing the patient to leave. The administrator must note symptoms such as cough, chest tightness, or anything unexpected. It is desired that the administrator leaves 5 mL of ozone in the syringe to keep the syringe from sucking in residual bodily fluids of the patient. The syringe should be reusable until a black ring inside the plunger become visible, or if the plunger gets too stiff to move it, or if the graduation markings fade and become obscure. It is also preferable that the administrator switch veins and arms being cannulated on a particular treatment session to allow the veins to recuperate and avoid repeated trauma to the same veins.

In regards to the side effects associated to the present invention, a cerebral paresis has been observed by the inventor in 1 out of 10,000 cases. A feeling of paresis may begin on one side of the patient's body within the first 30 minutes after the push starts. The feeling of paresis lasts from 2 to 30 minutes after the treatment and has not led to any residual effects. Cerebral paresis is only a sensation of weakness, not a related to actual physiological damage.

These side effects have never happened with the initial treatment session or during the actual treatment. If the patient has experienced cough or chest tightness, the dosage is not increased until he or she has the same dose again without any such experience.

In the rare event that distressing chest symptoms occur for the patient, oxygen is given at 3 litres per minute via the nasal cannula to speed the resolution of the chest discomfort. The patient is not discharged until he/she is well past the symptoms, and the patient is observed for 5 to 10 minutes after the symptoms disappear before patient leaves, whereupon the patient is breathing O₂ for the whole time.

For vein irritation, warm compresses every 10 minutes, Traumeel cream, and as a last resort, ibuprofen is applied to the cannulation area. To prevent vein irritation, the administrator makes sure that the patient is taking vitamin C to gut tolerance every 2 hours, 5 to 6 times a day. Also, the patient is made to drink 1 gallon of water everyday (if not contraindicated), and take at least 3 ounces of probiotic bio-juice daily to replenish bacteria in intestinal wall.

Although the invention has been explained in relation to its preferred embodiment, it is to be understood that many other possible modifications and variations can be made without departing from the spirit and scope of the invention as hereinafter claimed.

What is claimed is:

1. A treatment method to cure diseases within a patient by administering intravenous oxygen-ozone mixture, the method comprises the steps of:
(A) providing a syringe, an infusion device, and a tourniquet;
(B) identifying an accessible peripheral vein on an upper extremity of the patient;
(C) preparing a volumetric dosage of oxygen-ozone mixture with the syringe;
(D) applying the tourniquet to a cannulation area on the upper extremity and inserting the infusion device into the accessible peripheral vein;
(E) transferring the volumetric dosage of oxygen-ozone mixture from the syringe, through the infusion device, and into the accessible peripheral vein at a specified infusion rate by releasing the tourniquet from the cannulation area after a witnessed flash of blood;

2. The treatment method to cure diseases within a patient by administering intravenous oxygen-ozone mixture, the method as claimed in claim 1 comprises the steps of:
repeating steps (B) through (E) as a plurality of treatment sessions;
incrementally increasing the volumetric dosage of oxygen-ozone mixture by a specified volume during each treatment session;

3. The treatment method to cure diseases within a patient by administering intravenous oxygen-ozone mixture, the method as claimed in claim 2, wherein the volumetric dosage of oxygen-ozone mixture during an initial session is within a range of 10 to 20 milliliters (mL) and proportionately depends upon a weight and a habitus of the patient, and wherein the initial session is from the plurality of treatment sessions.

4. The treatment method to cure diseases within a patient by administering intravenous oxygen-ozone mixture, the method as claimed in claim 2, wherein the specified volume is approximately 10 mL.

5. The treatment method to cure diseases within a patient by administering intravenous oxygen-ozone mixture, the method as claimed in claim 2, wherein the volumetric dosage of oxygen-ozone mixture from a tenth session to a twelfth session is approximately 55 mL and proportionately depends upon a weight and a habitus of the patient, and wherein the tenth session to the twelfth session is from the plurality of treatment sessions.

6. The treatment method to cure diseases within a patient by administering intravenous oxygen-ozone mixture, the method as claimed in claim 1, wherein the infusion device is a 27-gauge infusion set and is in fluid communication with the syringe.

7. The treatment method to cure diseases within a patient by administering intravenous oxygen-ozone mixture, the method as claimed in claim 1, wherein the volumetric dosage of oxygen-ozone mixture is approximately 96% oxygen and 4% ozone.

8. The treatment method to cure diseases within a patient by administering intravenous oxygen-ozone mixture, the method as claimed in claim 1, wherein the specified infusion rate is approximately 1 mL every 5 to 15 seconds.

9. The treatment method to cure diseases within a patient by administering intravenous oxygen-ozone mixture, the method as claimed in claim 1, wherein the specified infusion rate is adjusted to be proportionately slower for a smaller diameter of the accessible peripheral vein.

10. The treatment method to cure diseases within a patient by administering intravenous oxygen-ozone mixture, the method as claimed in claim 1, wherein appendages of the patient are held straight during step (E).

11. The treatment method to cure diseases within a patient by administering intravenous oxygen-ozone mixture, the method as claimed in claim 1, wherein the patient is resting in a semi-recumbent position during step (E).

12. The treatment method to cure diseases within a patient by administering intravenous oxygen-ozone mixture, the method as claimed in claim 1 comprises the steps of:
detecting an obstruction for transferring the volumetric dosage of oxygen-ozone mixture during step (E);
stroking a proximal area on the upper extremity towards a heart of the patient in order to relieve the obstruction;

13. The treatment method to cure diseases within a patient by administering intravenous oxygen-ozone mixture, the method as claimed in claim 1 comprises the step of:
simultaneously executing a first iteration of steps (A) through (E) with a first syringe and a second iteration of steps (A) through (E) with a second syringe in order to double the volumetric dosage of oxygen-ozone mixture for a treatment session;

14. The treatment method to cure diseases within a patient by administering intravenous oxygen-ozone mixture, the method as claimed in claim 14, wherein the volumetric dosage of oxygen-ozone mixture doubles to a range between 110 mL to 120 mL.

15. The treatment method to cure diseases within a patient by administering intravenous oxygen-ozone mixture, the method as claimed in claim 1 comprises the step of:
prompting the patient to initiate a weight-lifting exercise in order to improve blood circulation around the cannulation area;
